(19) United States
(12) Patent Application Publication
(10) Pub. No.: US 2026/0124333 A1
(43) Pub. Date: May 7, 2026

(54) HEATING FRAGRANCE ATOMIZER WITH MULTIPLE FRAGRANCE OPTIONS

(71) Applicant: DONGGUAN YIH TEH ELECTRIC PRODUCTS CO., LTD., DongGuan City (CN)

(72) Inventor: Ming Jen Hsiao, Miaoli County (TW)

(21) Appl. No.: 18/988,782

(22) Filed: Dec. 19, 2024

Related U.S. Application Data
(63) Continuation-in-part of application No. 18/937,160, filed on Nov. 5, 2024.

(51) Int. Cl. A61L 9/03

(52) U.S. Cl. CPC A61L 9/035; A61L 9/037; A61L 2209/11; A61L 2209/12; A61L 2209/133; A61L 2209/135

(57) ABSTRACT

of the present invention has multiple mist outlets, heating devices and a rack with multiple assembly grooves and elastic clips, so as to be detachably combined with a plurality of fragrance liquid bottles. By controlling the function selection key of the control circuit of the fragrance atomizer, the selected liquid bottle can be heated and evaporated to produce fragrance. The fragrance can be changed at any time, with a variety of fragrance selection functions. The liquid bottles can be conveniently and quickly snapped into the respective elastic clips, or the liquid bottles can be easily pulled out of the respective elastic clips to be separated from the fragrance atomizer, and new liquid bottles can be conveniently replaced and then assembled into the respective elastic clips of the fragrance atomizer for use.

This application is a Continuation-in-Part of application Ser. No. 18/937,160 filed Nov. 5, 2024.

### BACKGROUND OF THE INVENTION

### 1. Field of the Invention

The present invention relates to a fragrance atomizer, and more particularly to a heating fragrance atomizer with multiple fragrance options.

### 2. Description of the Related Art

There are some problems with replacing the fragrance liquid bottle used in the fragrance atomizer. The user's hand takes the fragrance liquid bottle and assembles or removes it from the frame inside the atomizer, and the hand and the fragrance liquid bottle are easily impacted or interfered by the shell around the atomizer, or the fragrance liquid bottle is difficult to assemble on the frame, and the fragrance liquid bottle is difficult to be pushed out of the atomizer, or the shell of the atomizer is inconvenient and time-consuming to open, making the replacement of the fragrance liquid bottle slow and inconvenient.

Another problem is that fragrance atomizers can usually only produce one fragrance, and consumers cannot choose to change the fragrance.

Also, the fragrance atomizer uses a fragrance liquid bottle, and the essential oil or fragrance liquid absorbed by the core of the liquid bottle is consumed too much and too quickly due to excessive heating, resulting in a shorter realization of the fragrance, or the heating evaporation is poorly controlled, and the essential oil or fragrance liquid on the core cannot be effectively and appropriately heated to produce fragrance.

### SUMMARY OF THE INVENTION

In view of the above problems of the prior art, the present invention provides a heating fragrance atomizer with multiple fragrance options. The fragrance liquid bottles can be easily mounted or pushed out of the fragrance atomizer, and new fragrance liquid bottles can be easily and quickly replaced and then assembled in the fragrance atomizer for use.

The present invention also provides a heating fragrance atomizer with multiple fragrance selection functions.

The present invention further provides a heating fragrance atomizer with multiple fragrance options, which can provide appropriate and effective heating and a proper fragrance evaporation speed, and the fragrance atomizer can provide a long-lasting fragrance effect.

In some embodiments of the present invention, the heating fragrance atomizer with multiple fragrance options comprises a base, a lower shell, a rack, a control circuit, an electrical connection unit, a heating device, and an upper shell.

The lower shell is a hollow shell having a first opening on its upper side and a second opening on its lower side.

The rack comprises a support frame, and an assembly frame extending laterally from the top of the support frame. One side of the periphery of the assembly frame is horizontally recessed to form a first assembly groove. The support frame has a receiving groove on one side connected to the first assembly groove. The elastic clip is combined with the receiving groove. The heating device is combined with the upper side of the first assembly groove. The upper shell is provided with a mist outlet, and the upper shell is combined with the assembly frame and covers the heating device. The mist outlet, the heating device and the first assembly groove are interconnected. The free end of the support frame is connected to the first opening of the lower shell, and the second opening is connected to the base.

The control circuit and the electrical connection unit are combined in the lower shell, the electrical connection unit is electrically connected to the control circuit, and the control circuit is electrically connected to the heating device.

Thereby, the elastic clip is used to detachably connect with a liquid bottle. When the user holds the liquid bottle, the free end of the water-conducting core protruding from the mouth can easily pass through the first assembly groove to be disposed adjacent to the heating device, and the neck under the mouth can be quickly assembled in the first assembly groove from bottom to top. At the same time, the liquid bottle can be conveniently and quickly clamped in the elastic clip, or the liquid bottle can be easily pulled out of the elastic clip to be detached, and a new liquid bottle can be conveniently replaced and then assembled in the elastic clip of the fragrance atomizer for use.

In some embodiments of the heating fragrance atomizer with multiple fragrance options, multiple mist outlets, multiple heating devices, the upper shell with multiple first assembly grooves and multiple elastic clips are correspondingly assembled in the fragrance atomizer for detachably combining a plurality of liquid bottles. The consumer can thereby control, for example, a function selection key electrically connected to the control circuit, and can continuously press the function selection key to correspond to different liquids filled in any of the liquid bottles, so that aromatic liquids with different scents can be selectively heated and evaporated to produce fragrance, achieving a variety of aroma selection functions.

### BRIEF DESCRIPTION OF THE DRAWINGS

FIG. 1 is a three-dimensional external view of the heating fragrance atomizer with multiple fragrance options of the present invention.

FIG. 2 is an exploded view of FIG. 1.

FIG. 3 is an exploded view of FIG. 1 from another viewing angle.

FIG. 4 is a cross-sectional view of the internal frame and the heating device assembled with liquid bottles of the fragrance atomizer of FIG. 1 of the present invention.

FIG. 5 is a sectional view of FIG. 1.

### DETAILED DESCRIPTION OF THE PREFERRED EMBODIMENT

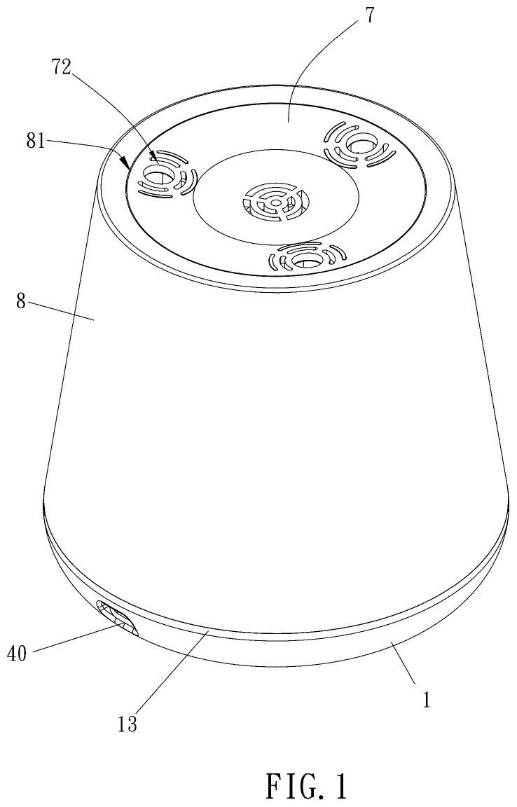

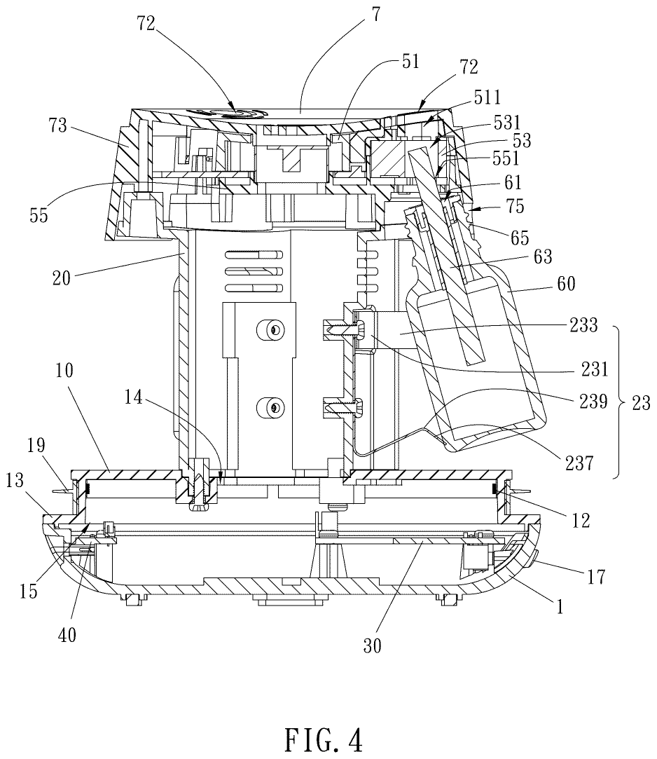

Please refer to FIGS. 1 to 4, some embodiments of a heating fragrance atomizer with multiple fragrance options according to the present invention are shown. The fragrance atomizer comprises a base 1, a lower shell 10, a rack 20, a control circuit 30, an electrical connection unit 40, a heating device 50, and an upper shell 7.

The lower shell 10 is a hollow shell having a first opening 14 on its upper side and a second opening 15 on its lower side.

The rack 20 comprises a support frame 21 and an assembly frame 22 extending transversely from the top side of the support frame 21. A first assembly groove 221 is horizontally recessed on one side of the periphery of the assembly frame 22. A receiving groove 211 connected to the first assembly groove 221 is provided on one side of the support frame 21, and an elastic clip 23 is coupled to the receiving groove 211.

The heating device 50 is combined with the upper side of the first assembly groove 221, and the upper shell 7 is provided with a mist outlet 72. The upper shell 7 is combined with the assembly frame 21 and covers the heating device 50. The mist outlet 72, the heating device 50 and the first assembly groove 221 are interconnected.

The lower free end of the support frame 21 is coupled to the first opening 14 of the lower shell 10, and the second opening 15 is coupled to the base 1.

The control circuit 30 and the electrical connection unit 40 are combined in the lower shell 10, and the electrical connection unit 40 is electrically connected to the control circuit 30 and the control circuit 30 is electrically connected to the heating device 50.

Thus, the elastic clip 23 is used to detachably engage a liquid bottle 60, such as a scented liquid bottle, a deodorant liquid bottle, or a bottled water bottle. The liquid bottle 60 comprises a bottle body 61, a water-conducting core 63, a neck 65, a mouth 67, a liquid (not shown), and a through bottle stopper 69. The liquid is filled in the bottle body 61. The neck 65 extends from the upper side of the bottle body 61. The mouth 57 is formed at the upper end of the neck 65. One side of the water-conducting core 63 is engaged in the mouth 67 to contact the liquid, and the other side of the water-conducting core 63 protrudes out of the mouth 67.

The water-conducting core 63 is, for example, a water-conducting fiber rod, which is made of, for example, PET, PP, TPU or TPEE and has good porosity to form a better water-conducting and water-absorbing capillary effect. When the user holds the liquid bottle 60, the free end of the water-conducting core 63 protruding from the mouth 61 can easily pass through the first assembly groove 221 to approach the heating device 50, and the neck 65 at the lower side of the mouth 61 can be quickly assembled from bottom to top corresponding to the first assembly groove 221. At the same time, the liquid bottle 60 can be conveniently and quickly clamped in the elastic clip 23, or the liquid bottle 60 can be easily pulled out of the elastic clip 23 to be detached, and a new liquid bottle 60 can be conveniently replaced and then assembled in the elastic clip 23 of the fragrance atomizer for use.

The electrical connection unit 40, such as a Type-C receptacle, an electrical plug or socket, or a USB plug or socket, is used to connect to a power source or a battery, etc., to provide power to the heating device 50 for generating a heat source for the liquid absorbed by the free end side of the water-conducting core 63, such as essential oil or scented liquid, to generate fragrance which is discharged into the environment through the mist outlet 72.

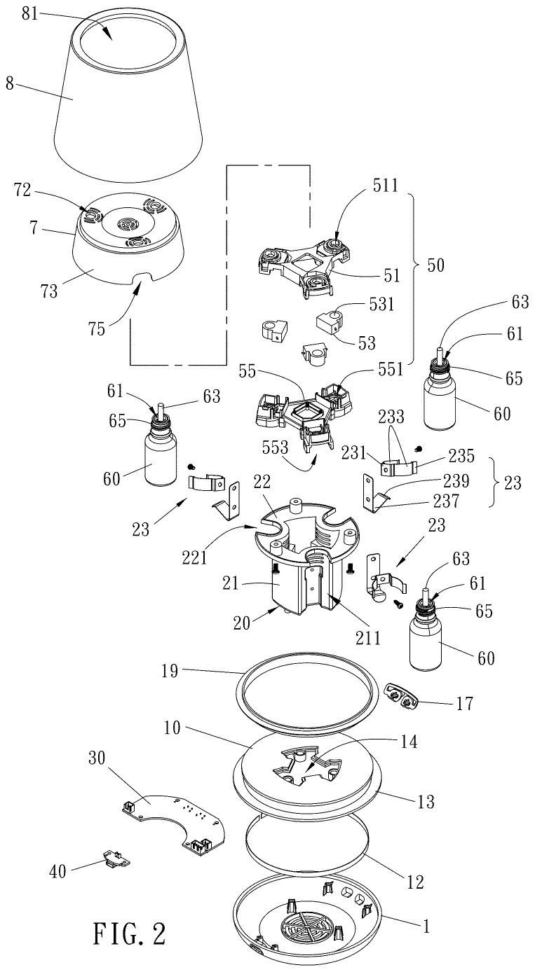

Referring to FIG. 1 and FIG. 2, an embodiment of the heating fragrance atomizer with multiple fragrance options of the present invention, the mist outlet 72 provided in the upper shell 7, the heating device 50, the first assembly groove 221 and the elastic clip 23 are respectively provided in the fragrance atomizer in a plurality of corresponding groups for detachably combining a plurality of liquid bottles 60. In the embodiment, there are three. The upper shell 7 is provided with three mist outlets 72 at intervals, the assembly frame 22 is provided with three first assembly grooves 221 at intervals, three heating devices 50 are provide to correspond to the three first assembly grooves 221, and the three mist outlets 72 correspond to the three heating devices 50, and the support frame 21 is provided with three receiving grooves 211 respectively connected to the three first assembly grooves 221, and three elastic clips 23 are respectively coupled to the three receiving grooves 211.

Thus, the consumer can use, for example, three liquid bottles 60 respectively combined in the elastic clips 23. Each liquid bottle 60 contains different liquids, such as lavender essential oil, chamomile essential oil or rosemary liquid, etc. The free end of the water-conducting core 63 protruding from the mouth 61 corresponds to the heating device 50 adjacent thereto. A function selection key 17 for operating the fragrance atomizer is electrically connected to the control circuit 30, and the function selection key 17 can be pressed continuously to select any liquid bottle 60 of a favorite fragrance. The heating device 50 generates heat to heat and evaporate the essential oil of the selected flavor absorbed by the free end of the water-conducting core 63 protruding out of the mouth 61, thereby achieving a variety of flavor selection functions.

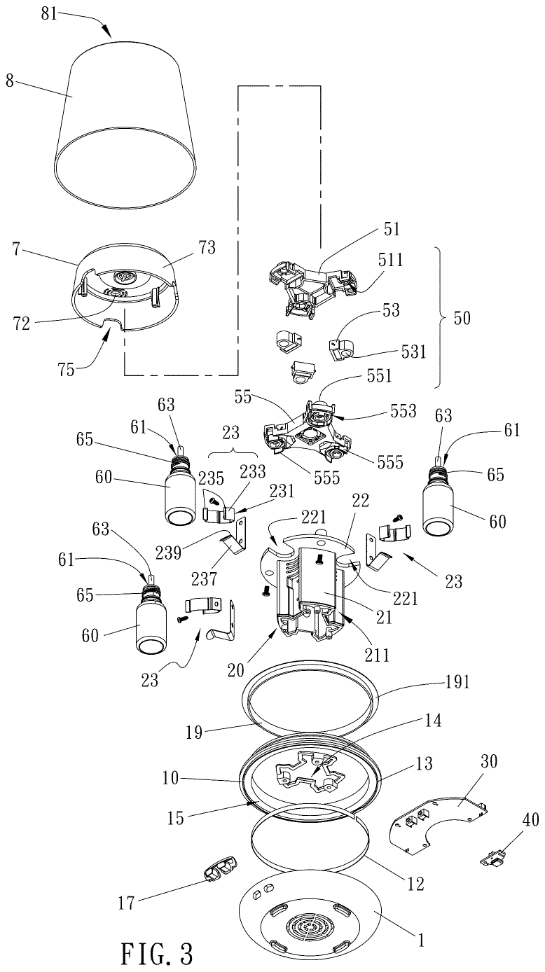

Referring to FIG. 2 and FIG. 3. In one embodiment of the heating fragrance atomizer with multiple fragrance options of the present invention, the elastic clip 23 comprises a clip base 231 and a pair of circularly curved arms 233. The pair of circularly curved arms 233 protrude from both sides of the clip base 231. A gap is left between the two free ends of the pair of circularly curved arms 233 for inserting a liquid bottle and clamping it on the elastic clip 23. The diameter of the pair of circularly curved arms 233 and/or the distance between the two free ends may be slightly smaller than the diameter of the liquid bottle 60, so as to preset the elasticity of the two circularly curved arms 233 in clamping the liquid bottle 60. The free ends of the pair of circularly curved arms 233 are respectively extended with guide pieces 235, and the guide pieces 235 are turned outward from the free ends of the pair of circularly curved arms 233 to allow the liquid bottle 60 to be easily inserted between the pair of circularly curved arms 233 and to be temporarily fixed in the pair of circularly curved arms 233 in a detachable manner.

In one embodiment of the heating fragrance atomizer with multiple fragrance options of the present invention, the essential oil or various fragrance liquids filled in liquid bottle 60 do not need high temperature to evaporate. The fragrance liquid or essential oil at the free end of water-conducting core 63 can be heated at a moderate to low temperature to prevent the essential oil or fragrance liquid in liquid bottle 60 from evaporating too much, consuming too quickly or having uneven fragrance concentration, so that the fragrance atomizer has a long-lasting fragrance effect, while taking heating safety and energy saving into consideration. The heating device 50 used generates a temperature of 50° C. to 70° C. to heat the essential oil. Some perfume liquids or perfumes are heated at a temperature of about 50° C. to 110° C. In some embodiments, if perfume oil is mixed with water, the temperature can be controlled at below 70° C. by using the heating device 50, where the water will not evaporate. The essential oil and perfume liquid at the free end of the water-conducting core 63 can be heated until they evaporate. On the other hand, the fragrance liquid at the free end of the water-conducting core 63 must be accurately and stably placed near the heating device 50 to receive medium to low temperature heating. With moderate and effective heating and a proper evaporation speed, the fragrance atomizer can emit fragrance for a long time.

In one embodiment of the ultrasonic atomizer of the present invention, the elastic clip 23 further comprises a spring 237, the spring 237 extends from the clip base 231 and is bent upward at an angle, the spring 237 is bent upward from the lower side of the clip base 231 for a distance, and one side of the free end of the spring 237 is further bent downward, and an elastic abutment portion 239 is formed at the bend.

When a liquid bottle 60 is assembled on the elastic clip 23, the bottom of the liquid bottle 60 presses the elastic abutment portion 239 downward, and the elastic abutment portion 239 is used to push the bottom of the liquid bottle 60 with the elastic force of the spring 237 to make the liquid bottle 60 upward. Thus, the mouth 61 can be tightly pressed against the bottom of the heating device 50, the liquid bottle 60 can be fixed without shaking, and the free end of the water-conducting core 63 can be accurately heated near the predetermined position of the heating device 50, for example, the free end of the water-conducting core 63 is heated in the middle of the heating device 50 or exactly close to the heating device 50 or in close proximity to the heating device 50. The essential oil of the flavor absorbed by the free end of the water-conducting core 63 can be effectively heated and evaporated at the correct position of the heating device 50.

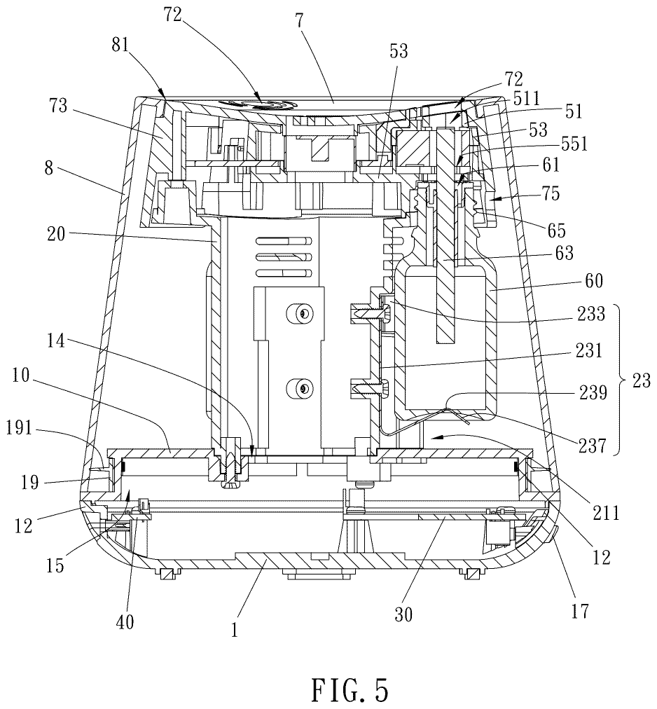

Please refer to FIGS. 2, 4 to 5, an embodiment of the heating fragrance atomizer with multiple fragrance options of the present invention is shown, wherein the heating device 50 comprises an upper cover 51, a heating element 53 and a bottom cover 55.

The upper cover 51 comprises a first hole 511, a heating hole 531 is formed in the middle of the heating element 53, and the bottom cover 55 comprises a second hole 551 and a second assembly groove 553 surrounding the lower side of the second hole 551. The heating element 53 is coupled to the second hole 551, and the upper cover 51 is coupled to the bottom cover 55, and covers the heating element 53 at the same time. The upper side of the heating hole 531 is connected to the first hole 511, and the lower side thereof is connected to the second hole 551. The bottom cover 55 is coupled to the assembly frame 21, and the second assembly groove 553 is coupled to the first assembly groove 221. On the other hand, the diameters of the second hole 551 and the heating hole 531 are slightly larger than the water-conducting core 63. The user picks up the liquid bottle 60 and passes the free end of the water-conducting core 63 through the second hole 551. It requires a certain degree of precision to correctly insert it into the heating hole 531. Therefore, the neck 65 of the liquid bottle 60 of the above-mentioned embodiment of the fragrance atomizer passes through the first assembly groove 221 from bottom to top, and the second assembly groove 553 can obviously guide the neck 65 of the liquid bottle 60 to the mouth 61, and can be assembled in the second assembly groove 553 in a fool-proof manner. The mouth 61 can be precisely assembled at the lower side of the second hole 551 to guide the free end of the water-conducting core 63 to accurately pass through the second hole 551 and be correctly inserted into the heating hole 531 to be surrounded by the heating element 53. The free end of the water-conducting core 63 is adjacent to the heating element 53, but the essential oil or fragrance oil absorbed by the water-conducting core 63 is heated through the air layer, so that the essential oil or fragrance oil and other fragrance liquids can be heated appropriately and effectively, and the fragrance can be evaporated appropriately, and the fragrance atomizer can emit fragrance for a longer time.

Please refer to FIG. 3, an embodiment of the heating fragrance atomizer with multiple fragrance options of the present invention is shown, the periphery of the second assembly groove 553 is formed by a plurality of spaced plates 555 surrounding the lower side of the second hole 551 to form the second assembly groove 553. The plates 555 are, for example, elastic materials, including any one of plastic sheets or metal sheets.

In some embodiments, the portion of the neck 65 to mouth 61 of the liquid bottle 60 is obliquely inserted upward into the second assembly groove 553 during the assembly process, and the elastic plates 555 at the periphery can be deformed by the contact and extrusion of the neck 65 and the mouth 61 of the liquid bottle 60 to overcome the obstruction of the neck 65 and the mouth 61 when they are assembled in the second assembly groove 553. Finally, the mouth 61 can be smoothly assembled at the lower side of the second hole 551, and guide the free end of the water-conducting core 63 to accurately pass through the second hole 551, and correctly penetrate into the heating hole 531 to receive heating.

Referring to FIGS. 2 to 4, in an embodiment of the present invention, the upper shell 7 comprises a top surface 71 and a side wall 73 extending downward from the periphery of the top surface 71. The mist outlet 72 is provided on the top surface 71, and a groove 75 is provided longitudinally at the bottom edge of the side wall 73. The upper shell 7 is combined with the assembly frame 22 and covers the heating device 50. The groove 75 corresponds to the outer side of the first assembly groove 221, so that the neck 65 of the liquid bottle 60 can easily pass through the groove 75 to be inserted into the first assembly groove 221, and the mouth 61 is assembled at the lower side of the second hole 551.

Referring to FIGS. 1 to 3 and 5, an embodiment of the heating fragrance atomizer with multiple fragrance options of the present invention further comprises an outer cover 8 having a top opening 81.

The lower shell 10 further comprises a rubber ring 19, and a protrusion 191 is formed on the outer periphery of the rubber ring 19. The rubber ring 19 is made of various elastic materials such as silicone, rubber, latex, etc. The rubber ring 19 is combined with the outer periphery of the lower shell 10. The bottom edge of the lower shell 10 further extends to have a ring protrusion 13. The outer cover 8 covers the heating device 50 and the rack 20. The top opening 81 is connected to the first hole 511. The bottom of the outer cover 8 is connected to the ring protrusion 13. At least a portion of the ring protrusion 13 is exposed between the outer cover 8 and the lower shell 10, and the inner edge of the bottom side of the outer cover 8 can be elastically abutted by the rubber ring 19 and its protrusion 191, so that the outer cover 8 can be detachably fixed to the lower shell 10. In the embodiment, the rubber ring 19 is made of silicone, and the protrusion 191 has a soft elastic deformation to press against the inner edge of the outer cover 8, and the outer cover 8 is detachably mounted on the outer periphery of the lower shell 10. On the contrary, the outer cover 8 can also be easily separated from the rubber ring 19 and the lower shell 10, so that the user can quickly and conveniently replace the liquid bottle 60 assembled in the elastic clip 23 of the fragrance atomizer, and the evaporated fragrance discharged from the first hole 511 is dissipated into the environment through the top opening 81.

Referring to FIGS. 2 to 5, an embodiment of the heating fragrance atomizer with multiple fragrance options of the present invention further comprises an LED light strip 12. The LED light strip 12 is electrically connected to the control circuit 30. The lower shell 10 is made of a light-transmitting material. The LED light strip 12 is integrated into the lower shell 10. The electrical connection unit 40 can connect a power source to the control circuit 30, so as to provide the LED light strip 12 with a strip, ring or intermittent point light source to the lower shell 10. The light source can be transmitted to the light-transmitting ring protrusion 13 exposed between the outer cover 8 and the lower shell 10 to produce light decoration or lighting effects. The light-transmitting material of the lower shell 10 can be any one of silicone, plastic, acrylic or glass.

In an embodiment of the heating fragrance atomizer with multiple fragrance options of the present invention, the function selection key 17 is electrically connected to the control circuit 30; the control circuit 30 comprises an electrically connected light emitting element 31, the function selection key 17 is combined with the outer side of the lower shell 10, the bottom side of the outer cover 8 is connected to the ring protrusion 13. On the other hand, the outer cover 8 can also be easily separated from the rubber ring 19 and the lower shell 10, and the user can quickly and conveniently operate the function selection key 17 to change the fragrance. The light emitting element 31, such as an LED, emits light of different colors inside the light-transmissive lower shell 10 to indicate the current operation option of the function selection key 17.

What the invention claimed is:

1. A heating fragrance atomizer with multiple fragrance options, comprising: a base, a lower shell, a rack, a control circuit, an electrical connection unit, at least one heating device an upper shell, wherein:
said lower shell is a hollow shell with a first opening on an upper side thereof and a second opening on an opposing lower side thereof;
said rack comprises a support frame, an assembly frame extending transversely from a top side of said support frame, at least one first assembly groove horizontally recessed on one side of the periphery of said assembly frame, at least one receiving groove provided on one side of said support frame and connected to said at least one first assembly groove, and at least one elastic clip coupled to said at least one receiving groove;
said at least one heating device is combined with an upper side of said first assembly groove;
said upper shell is combined with said assembly frame and covers said at least one heating device, said upper shell comprising at least one mist outlet interconnected with said at least one heating device and said at least one first assembly groove;
said support frame has a lower free end thereof coupled to said first opening of said lower shell, and the second opening is coupled to the base;
said control circuit is combined with said electrical connection unit in said lower shell, and is electrically connected to said at least one heating device;
said electrical connection unit is electrically connected to said control circuit.

2. The heating fragrance atomizer with multiple fragrance options as claimed in claim 1, further comprising at least one liquid bottle, each said liquid bottle comprising a bottle body, a water-conducting core, a neck, a mouth, a liquid and a through bottle stopper, said liquid being filled in said bottle body, said neck extending from an upper side of said bottle body, said mouth being formed at an upper end of said neck, said water-conducting core having one side thereof engaged in said mouth to contact said liquid and an opposite side thereof protruding out of said mouth.

3. The heating fragrance atomizer with multiple fragrance options as claimed in claim 1, wherein said at least one mist outlet, said at least one heating device, said at least one first assembly groove and said at least one elastic clip provided in said upper shell are respectively plural in number and are correspondingly assembled in the heating fragrance atomizer respectively to detachably combine the plurality of liquid bottles.

4. The heating fragrance atomizer with multiple fragrance options as claimed in claim 3, wherein said upper shell is provided with three said mist outlets at intervals, said assembly frame is provided with three said first assembly grooves at intervals, three said heating devices are provide to correspond to the three said first assembly grooves, and the three said mist outlets correspond to the three said heating devices, and said support frame is provided with three said receiving grooves respectively connected to the three said first assembly grooves, and three said elastic clips are respectively coupled to the three said receiving grooves.

5. The heating fragrance atomizer with multiple fragrance options as claimed in claim 1, further comprising at least one liquid bottle, each said liquid bottle comprising a bottle body, a water-conducting core, a neck, a mouth, a liquid and a through bottle stopper, said liquid being filled in said bottle body, said neck extending from an upper side of said bottle body, said mouth being formed at an upper end of said neck, said water-conducting core having one side thereof engaged in said mouth to contact said liquid and an opposite side thereof protruding out of said mouth, wherein each said elastic clip comprises a clip base and a pair of circularly curved arm respectively protruding from two sides of said clip base with a gap left between two free ends of said pair of circularly curved arms for inserting one said liquid bottle and clamping the inserted said liquid bottle on the respective said elastic clip, the diameter of said pair of circularly curved arms and/or the distance between the two free ends said pair of circularly curved arms being smaller than the diameter of the inserted said liquid bottle, so as to preset the elasticity of the two circularly curved arms in clamping the inserted said liquid bottle, the said free ends of said pair of circularly curved arms being respectively extended with a respective guide piece, each said guide piece being turned outward from the free end of the respective said circularly curved arm.

6. The heating fragrance atomizer with multiple fragrance options as claimed in claim 5, wherein each said elastic clip further comprises a spring, said spring extending from said clip base and bending upward at an angle, said spring bending upward for a distance from a lower side of said clip base, said spring having one side of a free end thereof bending downward again with the bending position forming the said elastic abutment portion of the respective said elastic clip.

7. The heating fragrance atomizer with multiple fragrance options as claimed in claim 6, wherein each said liquid bottle is assembled on one respective said elastic clip with the bottom thereof pressed on the said elastic abutment portion of the said elastic clip, and the said elastic abutment portion is used to press against the bottom of the said liquid bottle with the elastic force of the associating said spring to make the said liquid bottle face upward, whereby the mouth of the said liquid bottle is pressed tightly against a bottom of the respective said heating device and the said liquid bottle is fixed.

8. The heating fragrance atomizer with multiple fragrance options as claimed in claim 7, wherein said water-conducting core of each said liquid bottle has a free end thereof disposed adjacent to the respective said heating device, so that the free end of the said water-conducting core is heated in the middle of the respective said heating device or exactly close to the respective said heating device or closely adjacent to the respective said heating device.

9. The heating fragrance atomizer with multiple fragrance options as claimed in claim 6, wherein each said heating device comprises an upper cover, a heating element and a bottom cover, said upper cover comprising a first hole, said heating element having a heating hole formed in the middle thereof, said bottom cover comprising a second hole and a second assembly groove surrounding a lower side of said second hole, said heating element being coupled to said second hole, said upper cover being coupled to said bottom cover to cover said heating element, said heating hole having an upper side thereof connected to said first hole and a lower side thereof connected to said second hole, said bottom cover being coupled to said assembly frame, said second assembly groove being coupled to one respective said first assembly groove.

10. The heating fragrance atomizer with multiple fragrance options as claimed in claim 9, wherein said second assembly groove has the periphery thereof formed by a plurality of spaced plates surrounding a lower side of the associating said second hole to form the respective said second assembly groove.

11. The heating fragrance atomizer with multiple fragrance options as claimed in claim 10, wherein said plates are selectively made of elastic plastic or metal.

12. The heating fragrance atomizer with multiple fragrance options as claimed in claim 9, wherein upper shell is combined with said assembly frame and covers said at least one heating device, said upper shell comprising a top surface with said at least one mist outlet formed thereon, a side wall extending downward from the periphery of said top surface and at least one groove provided longitudinally at a bottom edge of said side wall, each said groove corresponding to an outer side of one respective said first assembly groove.

13. The heating fragrance atomizer with multiple fragrance options as claimed in claim 9, further comprising an outer cover having a top opening, wherein said lower shell further comprises a rubber ring and a light-transmitting ring protrusion extending from a bottom edge thereof, said ring protrusion having at least a part thereof exposed between said outer cover and said lower shell, said rubber ring having protrusion formed on the outer periphery thereof, said rubber ring being made of an elastic material selected from the group of silicone, rubber and latex, said rubber ring being combined with the outer periphery of said lower shell; said outer cover covers said at least one heating device and said rack with the said top opening thereof connected to the said first hole on the said upper cover of each said heating device, said outer cover having a bottom side thereof connected to said ring protrusion and an inner edge of the bottom side elastically abutted by said rubber ring and said protrusion, whereby said outer cover is detachably fixed to said lower shell.

14. The heating fragrance atomizer with multiple fragrance options as claimed in claim 13, further comprising a LED light strip combined inside said lower shell and electrically connected to said control circuit; said lower shell is made of a light-transmitting material; said electrical connection unit connects a power source to said control circuit to provide said LED light strip with a strip, ring or intermittent point light source to said lower shell, so that the light source is transmitted to the light-transmitting ring protrusion exposed between said outer cover and said lower shell.

15. The heating fragrance atomizer with multiple fragrance options as claimed in claim 14, further comprising a function selection key combined with an outer side of said lower shell and electrically connected to said control circuit; said control circuit comprises an electrically connected light emitting element; said outer cover has a bottom side thereof connected to the said ring protrusion and covers said function selection key.
